(19) United States
(12) Patent Application Publication
(10) Pub. No.: US 2004/0247654 A1
(43) Pub. Date: Dec. 9, 2004

(54) Hydrophilic adhesives for delivery of herbal medicines

(75) Inventors: Robert A. Asmus, Hudson, WI (US); Kejian Chen, Woodbury, MN (US); JingJing Ma, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/456,810

(22) Filed: Jun. 5, 2003

(51) Int. Cl. A61K 9/70

(52) U.S. Cl. 424/449

(57) ABSTRACT

A hydrophilic, pressure-sensitive adhesive composition comprising a swellable adhesive polymer, a swelling agent, herbal medicines, and optionally a modifying polymer in an amount sufficient to form a cohesive, pressure-sensitive adhesive composition. The composition is useful as a delivery device for herbal medicine and other active ingredients to or through skin. A method of preparation of the composition is also disclosed.

### FIELD OF THE INVENTION

This invention relates to a hydrophilic adhesive composition for delivery of herbal medicine. This invention also relates to articles made with the adhesive composition and a method for producing the adhesive composition.

### BACKGROUND OF THE INVENTION

Largely a product of Asian countries, and often referred to as “traditional Chinese medicine,” herbal medicines are prepared to address a variety of elements, and extensive research has been done to identify the active ingredients that produce the desired effect, such as anti-inflammation, pain reduction, and pain elimination. Both the active ingredient or ingredients, and their interaction with each other or the surface treated, remain unknown for many available herbal medicine preparations. However, these herbal ingredients have been used for centuries and their effectiveness has been widely accepted.

While most herbal medicines are administered orally, many herbal medicines can be administered for both topical and transdermal treatment. The concentrations of active agents in the herbal ingredients can be low or impure, which may result in high levels of loading in a delivery device to effect a therapeutic benefit.

Most existing delivery devices for herbal medicines are plasters of natural rubber-based adhesives. Natural rubber-based adhesives are hydrophobic, and have poor compatibility with hydrophilic herbal ingredients. The natural rubber-based adhesives are mixed with the herbal ingredients in significant amounts, and coated on a cloth or nonwoven backing. The plasters of herbal medicine have undesirable qualities such as irritation, poor adhesion, bulkiness, and minimal wear times of less than one day. Hydrogels made of hydrophilic polymers have only recently been used with traditional Chinese medicines to reduce irritation, and continue to have poor adhesive properties and limited ability to dissolve the herbal ingredients.

Increasing cohesiveness of hydrophilic adhesive compositions has been accomplished by crosslinking polymeric material. Crosslinking can be physical (thermally reversible in case of thermoplastics and thermoplastic elastomers) or chemical (permanent). Depending on the polymer system chosen, crosslinking may affect both the cohesive and adhesive aspects of the adhesive composition. Pressure-sensitive adhesives produced using multifunctional crosslinkers or radiation induced crosslinking can range in adhesive and cohesive character. In general, as the crosslinker concentration is optimized, an increase in cohesiveness and adhesiveness is achieved. Increasing or decreasing the crosslinker beyond the optimal concentration for a given system typically further reduces both cohesiveness and adhesiveness. This optimal characteristic can limit the ability to tailor the adhesive and cohesive needs for a given application.

Physical characteristics of the additives can also affect the cohesive and adhesive characteristics of the adhesive composition. While crosslinking to form a hydrogel adhesive has allowed for increased amounts of additives to an adhesive gel composition, the significant additive load required with herbal medicines can reduce cohesiveness of the composition below acceptable levels.

Balanced against the need for improved cohesiveness, a continuing concern exists for biocompatibility in the preparation of hydrophilic polymers used as skin adhesives. Not only must the pressure-sensitive adhesive composition adhere to skin, but also the adherence should not cause skin irritation, toxicity reaction, or other deleterious effects of contacting a polymeric composition to living tissue.

A need exists to increase the cohesive nature and absorptive swelling capacity of hydrophilic adhesive compositions while maintaining a low modulus, conformability, and gentle-to-skin adhesiveness, and retaining cohesiveness in the presence of additives such as herbal medicines or other therapeutic agents.

### SUMMARY OF THE INVENTION

The present invention provides hydrophilic, medically useful, pressure-sensitive adhesive compositions with optimal adhesive and cohesive properties. The adhesive composition comprises an adhesive polymer, a swelling agent; a herbal medicine and optionally, a modifying polymer swellable in the swelling agent, wherein the adhesive polymer forms a pressure sensitive adhesive when swelled by the swelling agent, and the combination of the modifying polymer and the swelling agent regulates the adhesiveness of the first polymer while increasing the cohesion of the composition.

In one aspect of the invention, the increased levels of swelling agent provide the ability to solubilize the active from the herbal medicine to increase herbal release activity.

In another aspect of the invention, the adhesive composition further comprises an antimicrobial agent and/or a therapeutic agent.

### BRIEF DESCRIPTION OF THE DRAWINGS

FIG. 1 is a top plan view of a medical dressing containing the adhesive composition of the present invention.

FIG. 2 is a side view of a medical dressing containing the adhesive composition of the present invention.

FIG. 3 is a graph showing release of herbal medicine from an adhesive composition prepared with polymers of the present invention.

FIG. 4 is a graph comparing release of herbal medicine at different loading levels in adhesive compositions prepared with polymers of the present invention compared to release of herbal medicine from commercially available herbal plasters.

FIG. 5 is a graph showing release of herbal medicine from an adhesive composition prepared with polymers of the present invention.

### DETAILED DESCRIPTION OF THE INVENTION

“Solid” means that poly(vinyl lactam) is not required to be mixed with any other material prior to irradiation to crosslink such poly(vinyl lactam). No mixing with solvents, swelling agents or chemical crosslinking agents is required to prepare radiation-crosslinked poly(vinyl lactam) useful for the present invention. Commercially available non-crosslinked poly(vinyl lactam) can be employed in particulate form for irradiation to crosslink such poly(vinyl lactam).

“Essentially unirradiated” means that additives useful with solid, radiation-crosslinked poly(N-vinyl lactam) is neither subjected to any irradiation during the crosslinking of such solid poly(N-vinyl lactam) nor is subjected to any irradiation at any other time at a dosage which would degrade the additives.

“Swelling agent” defined as a nontoxic substance capable of swelling polymer.

“Modifying polymer” defined as a polymer that, in the presence of the swelling agent, can maintain or increase cohesiveness.

As used herein, “herbal medicines” are herbal ingredients or mixtures with believed, suspected or known medicinal or therapeutic benefits. Herbal ingredients include materials derived from plant, animal, mineral and other sources. The ingredients of traditional Chinese medicines are included within the definition of “herbal medicine.”

“Pressure sensitive adhesives” (PSAs) are well known to one of ordinary skill in the art to possess properties including the following: (1) aggressive and permanent tack, (2) adherence with no more than finger pressure, (3) sufficient ability to hold onto an adherend, and (4) sufficient cohesive strength to be removed cleanly from the adherend. Materials that have been found to function well as PSAs include polymers designed and formulated to exhibit the requisite viscoelastic properties resulting in a desired balance of tack, peel adhesion, and shear holding power.

This invention utilizes a blend of a crosslinked poly vinyl lactam or other swellable polymer that forms a pressure sensitive adhesive upon swelling by a swelling agent, a swelling agent, herbal medicines and optionally, a swellable polymer that modifies the cohesiveness of the hydrogel when swelled by the swelling agent.

The adhesive composition could also be utilized to deliver other therapeutic agents, such as antimicrobial or pharmaceutical agents, onto or through the skin. Penetration enhancing agents or excipients could be added when a pharmaceutical or active agent for topical or transdermal delivery is desired. Additives to adjust the pH, buffer the pH, alter the ionic strength of the adhesive composition as well as pigments to alter the opacity, color, reflectivity or strength of the gel is also considered.

Swellable Adhesive Polymers

The adhesive composition of the present invention comprises a swellable polymer that forms a pressure sensitive adhesive upon swelling, a swelling agent, herbal medicines and optionally a modifying polymer present in an amount sufficient to form a cohesive, pressure sensitive adhesive composition. The amount of swelling agent to be mixed with the swellable polymer typically can range from about 50 to about 90 weight percent of the composition. Consequently, exclusive of any biocompatible and/or therapeutic materials to be added to the composition, the weight percent of the swellable polymer can be from about 10 to about 50 weight percent. When the swellable polymer is poly(N-vinyl lactam), the weight percent of poly(N-vinyl lactam) can range from about 15 to about 45 percent.

Suitable swellable adhesive polymers for use in the present invention include as polyethyleneoxide, poly (N-vinyl) lactam polymers, polyacrylamide, maleic anhydride-vinyl ether copolymers, polyacrylic acid, ethylene-maleic anhydride copolymers, polyvinyl ether, polyethyleneimine, polyvinyl alkyl pyridinium halides, polymethacrylic acid and copolymers and blends of the above. Other suitable hydrophilic polymers for use in the present invention are described in U.S. Pat. Nos. 2,838,421 (Sohl et al.); 4,413,080 (Blake et al); 3,865,770 (Blake et al); Re 34279 (Blake et al); 4,539,996 (Engel et al.); and 4,273,135 (Larimore et al); all of which are incorporated herein by reference. The polymers may be uncrosslinked or lightly crosslinked chemically, by radiation, or by other means known in the art, including those discussed in U.S. Pat. Nos. 5,409,966 (Duan et al); 4,931,282 (Asmus et al.); and 4,539,996 (Engel et al); all of which are incorporated herein by reference.

In some embodiments, the adhesive composition of the present invention comprises a swellable, crosslinked poly(N-vinyl lactam) combined with essentially unirradiated swelling agent, herbal medicines and optionally a modifying polymer present in an amount sufficient to form a cohesive, pressure-sensitive adhesive composition. When the poly(N-vinyl lactam) is poly(N-vinyl pyrrolidone), the weight percent of poly(N-vinyl pyrrolidone) can range from about 15 to about 45 percent and preferably from about 18 percent to about 35 percent.

In most embodiments using poly (N-vinyl lactam), the swellable, poly(N-vinyl lactam) is radiation-crosslinked, while the lactam is in a solid form. In other embodiments, the poly (N-vinyl) Lactam is crosslinked by free-radical polymerization, either in bulk or in solution, of a precursor containing an N-vinyl lactam monomer, optionally other monomers, and a crosslinking compound as described in U.S. Pat. No. 4,931,282, which is incorporated herein by reference.

Poly(N-vinyl lactam) useful in the present invention can be provided in any form susceptible to being crosslinked such as the solid forms described in U.S. Pat. Nos., 4,931,282, 5,225,473 and 5,389,376, the disclosures of which are incorporated in their entirety herein by reference thereto. Nonlimiting examples of solid forms include particles, pellets, sheets, flakes, and bulk objects of various shapes, and coated objects of various shapes. Typically, the poly(N-vinyl lactam) is in the form of particles of a size less than about 1 cm in diameter, more typically from about 0.1 micron to 0.250 cm and often from about 10 microns to about 1000 microns. Alternatively, the poly (n-vinyl) lactam can be crosslinked in solution. Poly(N-vinyl lactam) can be a noncrosslinked homopolymer or a noncrosslinked copolymer containing N-vinyl lactam monomeric units, which after irradiation becomes swellable in a swelling agent and is biocompatible with mammalian (e.g., human) skin. In most embodiments, a noncrosslinked homopolymer or noncrosslinked copolymer of poly (N-vinyl) lactam may be used which is soluble in a biocompatible swelling agent. Nonlimiting examples of N-vinyl lactam monomers are N-vinyl-2-pyrrolidone; N-vinyl-2-valerolactam; N-vinyl-2-caprolactam; and mixtures of any of the foregoing. Preferably, the N-vinyl lactam is N-vinyl-2-pyrrolidone. Typically, the poly(N-vinyl lactam) is a homopolymer of N-vinyl-2-pyrrolidone.

Nonlimiting examples of comonomers useful with the aforementioned N-vinyl lactam monomers include N,N-dimethylacrylamide, acrylic acid, methacrylic acid, hydroxyethylmethacrylate, acrylamide, 2-acrylamido-2-methyl-1-propane sulfonic acid or its salt, and vinyl acetate. Normally, N-vinyl lactam monomeric units will comprise no less than about 50 weight percent of the monomeric units present in the poly(N-vinyl lactam) in solid state form. Typically, N-vinyl lactam monomeric units comprise a majority of total monomeric units of the polymer, and more typically, the N-vinyl lactam monomeric units comprise 70 to 100 percent by weight of the poly(N-vinyl lactam) and often 90 to 100 percent by weight of the poly(N-vinyl lactam).

Noncrosslinked N-vinyl lactam homopolymer and N-vinyl pyrrolidone/vinyl acetate copolymers are commercially available. Nonlimiting sources of commercially available poly(N-vinyl pyrrolidone) useful for the present invention include Aldrich Chemical Co. of Milwaukee, Wis., BASF of Parsippany, N.J., ISP (GAF) of Wayne, N.J., Dan River Corporation of Danville, Va., and Spectrum Chemical Manufacturing Corporation of Gardena, Calif. Poly(N-vinyl lactam) can have a Fikentscher K-value of at least K-15, and normally at least K-60 more often K-90, or even K-120. Other Fikentscher K-values are possible. Fikentscher K-values are described in Molyneaux, Water-Soluble Polymers: Properties and Behavior, Vol. 1, CRC Press, 1983, pp. 151-152.

After exposure to ionizing radiation, poly(N-vinyl lactam) can have a Swelling Capacity in water of at least about 15, typically at least about 30, and often at least about 40 as described in U.S. Pat. No. 5,409,966, which is incorporated herein by reference.

Swellable Modifying Polymers

Optionally, a modifying polymer is added to the adhesive composition to improve the cohesive characteristics of the adhesive composition. The modifying polymer is present in the adhesive composition to maintain and/or increase cohesiveness while reducing adhesiveness. When added with the swelling agent, the modifying polymer becomes solublized or suspended in the swelling agent. Typically, the modifying polymer will form a viscous solution or viscous gel when combined with the swelling agent in a ratio of modifying polymer to swelling agent of 1:9.

The choice of swelling agent typically will determine the appropriate modifying polymer to accomplish maintaining or improving cohesion of the adhesive composition. Modifying polymers that are poorly solubilized in one swelling agent may be highly swollen in a different swelling agent for use in the present invention. Modifying polymers useful in the present invention are further described in co-pending co-assigned patent application, “Adhesive Compositions, Articles Incorporating Same and Methods of Manufacture,” Attorney Docket number 58649US002.

Examples of suitable modifying swellable polymers include a polysaccharide, polysaccharide derivatives, acrylate derivates, collagen, collagen derivatives, cellulose, cellulose derivatives, polyvinyl alcohols and combinations thereof. In particular embodiments, modifying swellable polymers for use in the present invention are hydroxypropyl guar; guar gum; hydroxyethyl cellulose; hydroxypropyl cellulose; hydroxypropyl methylcellulose; polymeric quaternary ammonium salt of hydroxyethyl cellulose reacted with trialkyl ammonium substituted epoxide; copolymers of hydroxyethyl cellulose and diallyldimethyl ammonium chloride; and derivatives and combinations of the foregoing.

Swelling Agents

Hydrophilic, pressure-sensitive adhesive compositions of the present invention contain a swelling agent to swell the adhesive polymer and the modifying polymer. The swelling agent can be any swelling agent which can swell both the adhesive polymer and the modifying polymer and which is biocompatible with skin.

Nonlimiting examples of swelling agents useful to swell the polymers of the adhesive composition include monohydric alcohols (e.g., ethanol and isopropanol), polyhydric alcohols, (e.g., ethylene glycol, propylene glycol, polyethylene glycol (Molecular Weight between 200 and 600) and glycerin), ether alcohols (e.g., glycol ethers), other polyol swelling agents which do not cause skin irritation or toxic reaction, and water.

Depending on the ultimate use desired for the adhesive composition, non-volatile and/or volatile swelling agents may be used. One suitable swelling agent may comprise volatile swelling agent and non-volatile swelling agent, such as a mixture of glycerin or polyethylene glycol with water. In some embodiments, non-volatile swelling agents may be used by themselves such as, for example, glycerin or polyethylene glycol. Likewise, volatile swelling agents such as water may be used alone in the compositions of the invention. For this invention, “essentially non-volatile” means that a swelling agent as used in the present invention will render the adhesive polymer, such as radiated poly(N-vinyl lactam), sufficiently cohesive and pressure sensitive adhesive, such that less than ten percent (10%) of a given volume of swelling agent evaporates after exposure to processing or storage conditions.

The swelling agent can be added in an amount ranging from about 50 to about 90 weight percent of the adhesive composition and preferably from about 60 to about 80 weight percent. In one embodiment, the essentially non-volatile swelling agent, glycerin is chosen as the essentially non-volatile swelling agent. In most embodiments, the non-volatile swelling agent is present greater than 30% in the adhesive composition.

Other non-limiting examples of swelling agents which would be useful include monohydric alcohols, (e.g. ethanol, isopropanol, n-propanol), polyhydric alcohols (propylene glycol, dipropylene glycol, polyethylene glycol (PEG-2 to PEG-45M, preferably of molecular weight between 200 and 600) glycerol, polyglycerols (e.g. diglycerin, triglycerol, polyglycerin-3, hexaglycerol and decaglycerol), sorbitol and polyhydric alcohol ethoxylates (e.g. sorbeth-6, sorbeth-30, glycereth-1 to glycereth-31) methoxides of polyethylene glycol (Methoxy PEG-2 to Methoxy PEG 100), methoxides of polyhydric alcohol ethoxylates (e.g. glycereth-7 methoxide).

The swelling agent is typically a liquid. In some embodiments, humectant-type solid swelling agents like sorbitol could be used in conjunction with a co-swelling agent in order to dissolve and remain as a liquid. Other humectants that could also be employed as swelling agents or co-swelling agents include: 1,2,6-hexanetriol, acetamide mea, aluminum hydroxide, arginine pca, butoxypropanol, butylene glycol, dimethyl imidazolidinone, dimethylsilanol hyaluronate, dipotassium glycyrrhizate, erythritol, ethoxydiglycol, fructose, glucamine, gluconic acid, glucose, glucose glutamate, glucuronic acid, glutamic acid, glycogen, glycyrrhizic acid, heilmoor clay, hexacosyl glycol, histidine, hyaluronic acid, hydrogenated honey, hydrogenated starch, hydrolysate, hydrolyzed collagen, hydrolyzed elastin, hydrolyzed glycosaminoglycans, hydrolyzed keratin, hydrolyzed silk, hydrolyzed soy protein, hydrolyzed wheat protein, hydroxyethyl sorbitol, inositol, inositol hexa-pca, lactamide mea, lactic acid, lactitol, lactose, lysine pca, magnesium pca, maltitol, manganese pca, mannitol, mel (honey extract), menthyl pca, methyl gluceth-10, methyl gluceth-20, pca (pidolic acid), lactamide, polydextrose, polyglucuronic acid, polyglyceryl sorbitol, potassium pca, ppg-20 methyl glucose ether, ppg-38-buteth-37, saccharide isomerate, serica, silk amino acids, sodium carboxymethyl chitin, sodium lactate, sodium mannuronate methylsilanol, sodium pca, sodium pca methylsilanol, sodium polyglutamate, soluble collagen, sorbitol, sucrose, tea-lactate, tea-pca, trehalose, trilactin, urea, xylitol, zea mays, zinc pca, and combinations thereof.

Biocompatible and/or Therapeutic Additives

Depending upon the use of the hydrophilic, pressure-sensitive adhesive composition of the present invention, various other biocompatible and/or therapeutic materials can be included in the composition.

Hydrophilic, pressure-sensitive adhesive compositions of the present invention can also be used to deliver other pharmaceuticals to or through skin, such as topical or transdermal drug delivery systems. The pharmaceutical or other active ingredient can be compounded with the adhesive composition after poly(N-vinyl lactam) has been radiation-crosslinked, minimizing any possible deleterious interaction of the pharmaceutical or active ingredient with ionizing radiation in dosages sufficient to crosslink poly(N-vinyl lactam).

The hydrophilic, pressure-sensitive adhesive composition can also be used in therapeutic skin coverings, such as wound closure materials, tapes, and the like. For skin covering uses, other biologically active materials in addition to the herbal medicines can be added to the composition of the present invention. Nonlimiting examples of such other biologically active materials include broad spectrum antimicrobial agents where it is desired to reduce bacteria levels to minimize infection risk or treat the effects of infections at the skin or skin openings of a patient. Broad spectrum antimicrobial agents are disclosed in U.S. Pat. No. 4,310,509, which disclosure is incorporated by reference.

Other biocompatible and/or therapeutic materials can be added to the composition such as compounds to buffer the pH of the composition to provide a non-irritating pH for use with sensitive mammalian skin tissue or to otherwise maximize antimicrobial activity. Also, penetration enhancing agents or excipients can be added to the composition when the pharmaceutical or other active agent for topical or transdermal delivery so requires.

Irradiation Crosslinking of Poly(N-Vinyl Lactam)

Poly(N-vinyl lactam) in any solid form is subjected to ionizing radiation from a high-energy source. Nonlimiting examples of ionizing radiation include alpha, beta, gamma, electron-beam, and x-ray radiation. Of these sources of ionizing radiation, electron-beam irradiation and gamma irradiation are preferred. Sources of electron-beam radiation are commercially available, including an Energy Sciences Inc. Model CB-150 Electrocurtain Electron Beam Processor. Sources of gamma irradiation are commercially available from Atomic Energy of Canada, Inc. using a cobalt-60 high-energy source.

Ionizing radiation dosages are measured in megarads (mRad) or kilograys (kGy). Doses of ionizing radiation can be administered in a single dose of the desired level of ionizing radiation or in multiple doses which accumulate to the desired level of ionizing radiation. The dosage of ionizing radiation cumulatively can range from about 25 kGys to about 400 kGys and preferably from about 25 kGys to about 200 kGys. Preferably, ionizing radiation can achieve the desired level of crosslinking of poly(N-vinyl lactam) when the cumulative dosage of ionizing radiation exceeds 100 kGys (10 mRads).

Poly (N-vinyl lactam) can be irradiated in a solid form with ionizing radiation in a package or container where the temperature, atmosphere, and other reaction parameters can be controlled. One method of irradiating the Poly (N-vinyl lactam) in the present invention is described in U.S. Pat. No. 5,409,966, which is incorporated herein by reference. Depending upon the control of the irradiation conditions, poly(N-vinyl lactam) can be irradiated in a batch or continuous process.

Method of Preparing Hydrophilic Adhesive Compositions with Herbal Medicines

A method of preparing a pressure-sensitive adhesive composition of the present invention comprises mixing crosslinked poly(N-vinyl lactam) with a swelling agent and a modifying polymer, and herbal medicines in a solvent which is may be somewhat volatile at or above ambient temperatures. Typically, the swelling agent, modifying polymer, and herbal medicines are in essentially unirradiated form. Examples of suitable volatile solvents include water, ethanol, methanol, and isopropanol. A quantity of the resulting suspension is then cast onto a surface of a substrate, such as a release liner or a backing material and then stored. The volatile solvent is evaporated by heating such as by the application of microwave energy, infrared energy, or by convective air flow or the like, in order to form a cohesive, pressure-sensitive adhesive composition on the substrate. Often, a drying oven heated to about 65 degree C. may be employed for the evaporation step. A product release liner can optionally be laminated over the exposed surface of the composition to protect it from contamination.

In some embodiments, coating of the adhesive composition can be applied to the surface of a substrate. Suitable wet coating thicknesses may range from about 0.125 mm to about 1.25 mm so that, after evaporation of solvent, a dry coating thickness is obtained within the range from about 0.05 mm to about 0.38. Such coatings can be applied to any of a variety of substrate surfaces to act as an adhesive layer for the substrate and providing an adhesive composition with a low profile.

The method of preparing the compositions of the invention can be a batch process or a continuous line process. If prepared by a continuous process, the laminate of a liner, field of cohesive, pressure-sensitive adhesive composition, and substrate can be wound on roll for bulk packaging and further processing or can be cut using dies known to those skilled in the art into individual units.

Transdermal Delivery of Herbal Medicines

The adhesive composition of the present invention has demonstrated compatibility with a wide variety of herbal medicines, efficiently dissolves the herbal ingredients, while maintaining both adhesive and cohesive strength. The modifying polymers present in the adhesive composition provide greater cohesiveness than the hydrogel formed with the adhesive polymers alone, particularly for herbal ingredients of a grass, particulate or powder consistency. Additionally, the solubility of the herbal ingredients in the swelling agents can reduce the amount of herbal ingredients needed to produce the same effect. Thus, reduced levels of herbal ingredients can provide less bulk in the adhesive composition without any corresponding reduction in efficacy. Alternatively, greater efficacy can be accomplished without reducing the amount of herbal ingredients.

In an embodiment of the present invention, the adhesive composition comprises crosslinked poly n-vinylpyrrolidinone, a modified polymer of hydroxypropyl guar, glycerin as a swelling agent, water, and herbal medicines. Glycerin at high levels in the adhesive composition provides effective dissolution of many types of hydrophilic herb ingredients used as herbal medicine. This increased dissolution ability in an adhesive allows incorporation of increased herbal ingredient loads.

Solubility Study

Table 1 shows the solubility of herbal ingredients (available from Chee Zheng Tibetan Medicine Group, Tibet, China) in various ratios of glycerin to water. 0.1 g of herbal medicine was added to 10 mL of glycerin/water. The mixture was shaken for 8 hours. The solution was analyzed with an Agilent 1100 liquid chromatograph (Agilent technologies, Wilmington, Del.). Using the peaks from the HPLC analysis, solubility ratio was calculated as solubility in water divided by solubility in glycerin/water mixture.

TABLE 1
Herbal Medicine Solubility

| Glycerin Fraction in Water/Glycerin | Solubility Ratio |
|---|---|
| 0.00 | 1.00 |
| 0.10 | 1.29 |
| 0.20 | 1.95 |
| 0.30 | 2.41 |
| 0.40 | 3.17 |
| 0.50 | 4.20 |
| 0.60 | 5.04 |
| 0.70 | 5.08 |
| 0.80 | 4.74 |
| 0.90 | 4.44 |

The results indicated that the presence of glycerin or glycol compounds or oligomers increased the solubility of herbal medicines, particularly when added at high levels in the adhesive composition.

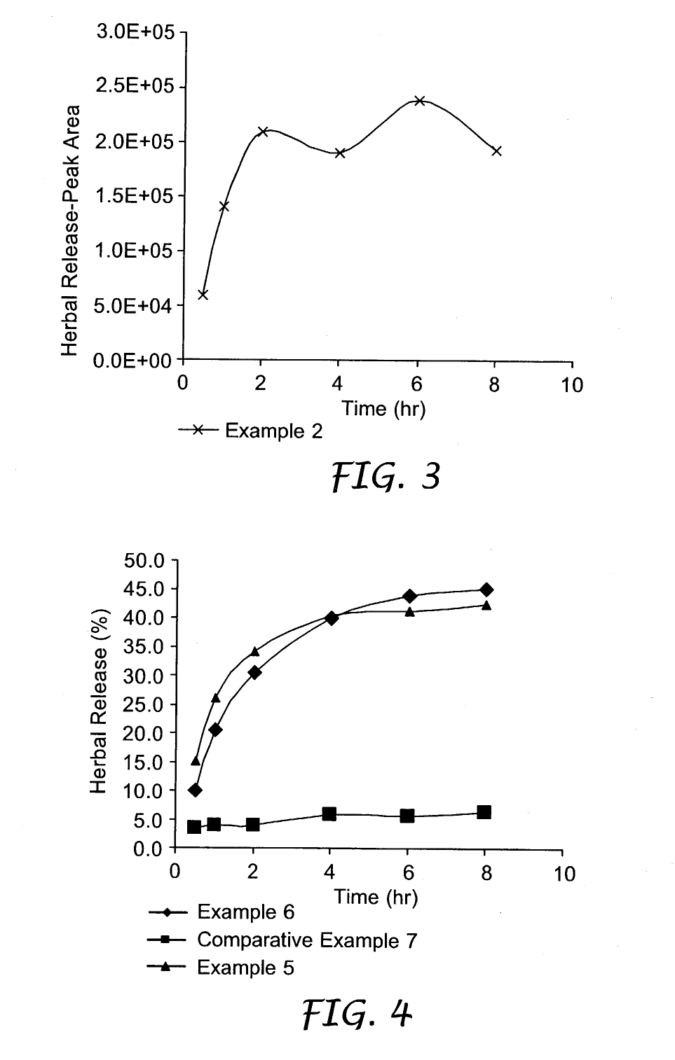

With increased solubility, the active ingredient of the herbal medicine is extracted more efficiently and demonstrates better release ability from the delivery device. Herbal release activity of herbal medicines in glycerol are shown in FIGS. 3-5 and discussed in the Examples below. The increased release ability of the adhesive compositions allows development of delivery devices or dressings containing herbal medicines with lower profiles for reduced bulk, more breathability and comfort for the wearer.

When added, the modifying polymer aids in retaining cohesiveness of the adhesive composition. The addition of herbal ingredients typically decreases cohesiveness based on the physical characteristics and quantity of the herbs. With the modifying polymer, the cohesiveness of the hydrogel can be preserved with minimal impact on adhesiveness when the herbal ingredients are added to the adhesive composition.

A wide variety of herbal medicines can be used in the present invention, including, but not limited to, Astragdi Radix, Atractylodis rhixoma, Ledebourellae Radix, Preparata Rehimanniae Radix, Comi Fructus, Dioscoreae Rhizoma, Alismatis Rhizoma, Moutan Radicis Cortex, Hoelen, Rehmanniae Radix, Dioscoreae rhizomma, Lycii Fructus, Corni Fructus, Cyanthulae Radix, Cuscutae Semen, Cornu cervi Colla, Plastrum Testudinis Colla, Sargasso Thallus, salvia, wild aconite root, frankincense, myrrh, nux vomica, cassia, tenuifolia, ledebouriella, Chinese silkvine root bark, drynaria rhizome, dahurian angelica root, resurrection lily rhizome, ginger, atractylodes rhizome, and other herbs as listed in U.S. Pat. No. 6,004,969. A more comprehensive listing of herbal medicines is provided in Chinese Herbal Medicine—Materia Medica, (Revised Ed. 1993) and The Pharmacology of Chinese Herbs, (2^{nd} Ed. 1999). In most embodiments, the herbal medicines are present up to 60% by volume in the present invention. In particular embodiments, the herbal ingredients are added at levels from 5 to 30 weight percent based on the total weight of the composition.

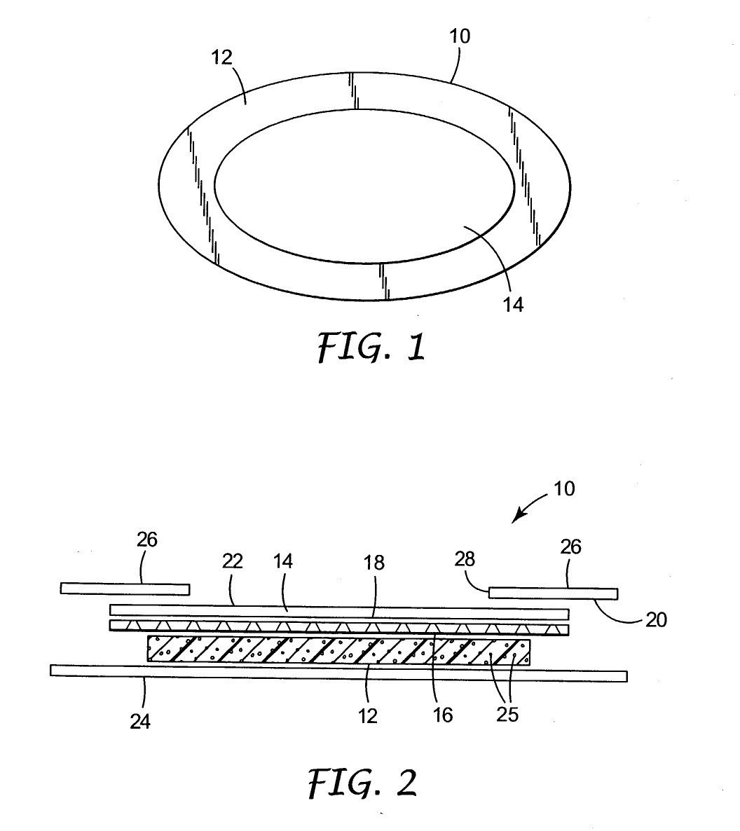

FIG. 1 shows a top plan view of a medical dressing 10 having a backing material 12, a layer 14 of pressure sensitive adhesive composition of the present invention coated on backing material 32. The medical dressing 10 is typically protected until use by a release liner, and optionally further includes a carrier delivery system. Although adhesive composition 12 is shown as centered on dressing 10, it can take any appropriate shape aid/or can be located off center on the dressing 10 as desired. Additionally, adhesive composition 14 could cover the surface of backing material 12.

Suitable dressing configurations for use in the present invention are disclosed in U.S. Pat. Nos. 6,436,432 (Heinecke et al); 6,264,976 (Heinecke et al); 5,976,117 (Dunshee et al); and U.S. Publication No. 2003/0007999 (Blatchford et al).

The adhesive layer 14 may be coated on a layer of backing material 12 selected from any of several backing materials having a high moisture vapor transmission rate for use as medical tapes, dressings, bandages, and the like. Suitable backing materials include those disclosed in U.S. Pat. Nos. 3,645,835 and 4,595,001, the disclosures of which are incorporated by reference. Other examples of a variety of films commercially available as extrudable polymers include “Hytrel™ 4056” and “Hytrel™ 3548” branded polyester elastomers available from E. I. DuPont de Nemours and Company of Wilmington, Del., “Estane” branded polyurethanes available from B. F. Goodrich of Cleveland, Ohio or “Q-thane” branded polyurethanes available from K.J. Quinn & Co. of Malden, Mass.

FIG. 2 depicts a side view of a particular embodiment of dressing 10 before placement on human skin. The adhesive composition 12 is positioned on a conformable backing 14 that is light and flexible relative to the composition 12. In most embodiments, a second pressure sensitive adhesive (PSA) 16 is provided on along one major surface 18 of the backing 14, and a low adhesion coating (low adhesion backsize or LAB) 20 is provided on the other major surface 22 of the backing 14.

Major surface 18 is sometimes referred to as the “bottom face” or “first major surface” of the backing, 14, and major surface 22 is sometimes referred to as the “top face” or “second major surface” of the backing 14. A release liner 24 is attached to the exposed surface of PSA 16 on the bottom face 18 of the backing 14. The release liner 24 covers the PSA 16 and the adhesive composition 12 until the consumer is ready to apply the dressing 10. The release liner 24 may be a single piece or multiple piece release liner, and may be part of or laminated to the package (not shown) containing the dressing, or merely enclosed along with the dressing 10 within the package.

The dressing 10 is sometimes referred to as an “island dressing” because the backing 14 extends substantially beyond the adhesive composition 12, typically beyond the entire periphery of the adhesive composition 12. A carrier frame 26 is attached to the top face 22 of the backing 14 over the low adhesion coating 20. The carrier frame 26 extends along substantially the entire periphery of the backing 14 and forms a window 28 exposing a portion of the backing 14 overlying the adhesive composition 12 with the backing 14 sandwiched between the frame 26 and adhesive composition 12.

Typically, herbal medicine 25 is contained in layer 12 by adding herbal medicine 25 to essentially unirradiated swelling agent or composition prior to coating on backing material 14. Alternatively, layer 12 can be used as a caulkable sealant according to U.S. Pat. No. 4,931,282 (Asmus et al.), the disclosure of which is incorporated by reference herein.

Hydrophilic, pressure-sensitive adhesive compositions of the present invention can be used as discrete gel particles dispersed in a continuous pressure-sensitive adhesive matrix to form a two phase composite useful in medical applications, as described in co-pending, co-assigned U.S. patent application Ser. No. 07/458,246, the disclosure of which is incorporated by reference herein.

The adhesive layer 34 can be coated on the backing layer 32 by a variety of processes, including, direct coating, lamination, and hot lamination. The release liner 36 can thereafter be applied using direct coating, lamination, and hot lamination.

The methods of lamination and hot lamination involve the application of pressure, or heat and pressure, respectively, on the layer of adhesive layer 12 to the backing material layer 14. The temperature for hot lamination ranges from about 50 degree C. to about 250 degree C., and the pressures applied to both lamination and hot lamination range from 0.1 Kg/cm² to about 50 Kg/cm².

For use, the release liner 24 is removed and the adhesive composition 12 can be applied to the skin of the patient as a part of a medical tape, a wound dressing, a bandage of general medicinal utility, or other medical device having water moisture absorbing properties. After placement on the patient, the carrier frame 26 can be removed.

Other medical skin coverings employing the hydrophilic, pressure-sensitive adhesive compositions of the present invention, optionally having antimicrobial and other biologically active agents contained therein, are useful for treatment of skin openings or wounds against the possibility of infection. The biologically active agent can be any therapeutically active material known to those skilled in the art and approved for delivery topically to or transdermally or iontophoretically through the skin of a patient. Non-limiting examples of therapeutic agents useful in transdermal delivery devices are any active drug or salts of those drugs, used in topical or transdermal applications, or growth factors for use in enhancing wound healing. Other therapeutic agents identified as drugs or pharmacologically active agents are disclosed in U.S. Pat. Nos. 4,849,224 and 4,855,294, and PCT Patent Publication WO 89/07951.

Excipients or penetration enhancing agents are also known to those skilled in the art. Non-limiting examples of penetration enhancing agents include ethanol, methyl laurate, oleic acid, isopropyl myristate, and glycerol monolaurate. Other penetration enhancing agents known to those skilled in the art are disclosed in U.S. Pat. Nos. 4,849,224; and 4,855,294 and PCT Patent Publication WO 89/07951.

A further description of the invention may be found in the following examples. All numbers are in weight percent unless specified otherwise.

### EXAMPLES

GLOSSARY OF TERMS

| Name | | |
|---|---|---|
| (Abbreviation) | Chemical Description | Source, Address |
| XPVP | Gamma crosslinked K-90D | 3M, St. Paul, MN (or |
| | polyvinylpyrrolidone | U.S. Pat. No. |
| | | 5,409,966) |
| Jaguar HP-120 | Hydroxypropyl Guar | Rhodia, Cranbury, NJ |
| | (HPG) | |
| Glycerin | 1,2,3-propanetriol | Dow Chemical Co., |
| | | Midland, Michigan |
| Huo Luo Xiao | Ingredients: Angelica | May Way Co. |
| Lin Wan | sinensis root, Salvia | Oakland, CA |
| | miltlorrhiza root, Boswellia | |
| | carterii resin, Commiphora | |
| | myrrha resin | |
| Yu Ping Feng San | Herbal ingredients: | Ming Tong Co., |
| (YPFS) | Astragdi Radix, | Taichung, Taiwan |
| | Atractylodis rhixoma, | |
| | Ledebourellae Radix.) | |
| Rhmannia Six | Ingredients: Preparata | Ming Tong Co., |
| Formula | Rehimanniae Radix, Comi | Taichung, Taiwan |
| | Fructus, Dioscoreae | |
| | Rhizoma, Alismatis | |
| | Rhizoma, Moutan Radicis | |
| | Cortex, Hoelen. | |
| Zuo Gui Wan | Ingredients: Rehmanniae | Ming Tong Co., |
| | Radix, Dioscoreae | Taichung, Taiwan |
| | rhizomma, Lycii Fructus, | |
| | Corni Fructus, Cyanthulae | |
| | Radix, Cuscutae Semen, | |
| | Cornu cervi Colla, | |
| | Plastrum Testudinis Colla | |
| Tang-kuei & | Ingredients: Evodiae | Ming Tong Co., |
| Evodia | Fructus, Angelicae Sinensis | Taichung, Taiwan |
| combination | Radix, Ligustici Wallichii | |
| | Rhizoma, Paeoniae Alba | |
| | Radix, Geneseng Radix, | |
| | Cinnamomi Ramulus, Asini | |
| | Gelatinum, Moutan Radicis | |
| | Cortex, Glycyrrhizae | |
| | Radix, Zingiberis Rhixoma, | |
| | Pinelliae Tuber, | |
| | Ophiopogonis Tuber. | |
| Seaweed | Sargasso Thallus | Ming Tong Co., |
| | | Taichung, Taiwan |
| Dan Seng | Salvia miltlorrhiza root | Bio Essence |
| | | Corporation, Richmond, |
| | | CA |
| Yun-Nan | Gum-like herbal mixture | Yun-Nam Bai-Yiao |
| Bai-Yiao | | Group Co., LTD, Yun- |
| (BY) | | Nan, China |
| Yun-Nan | Commercial brand plaster | Yun-Nam Bai-Yiao |
| Bai-Yiao | | Group Co., LTD, Yun- |
| plaster | | Nan, China |
| Chee Zheng (CZ) | Dried fine powders of | Chee Zheng Tibetan |
| | Tibetan herbals | Medicine Group, Tibet, |
| | | China |

### Examples 1-6

Herbal-hydrogel compositions were prepared using the components and amounts shown in Table 1. Examples 2-6 were prepared by premixing CZ herbal powder (Example 2-4) or BY herbs (Examples 5-6) with deionized water. The other components were weighed and mixed in a container at room temperature until a uniform past was formed. Example 1 was prepared the same way but without the herbal component. The pastes were poured onto and pressed for about 5 minutes between two release liners with a gauged thickness of 0.5 mm.

TABLE 1

| | Polymer | | | Glyc- | | | |
|---|---|---|---|---|---|---|---|
| Exam- | | Molec- | | erol | Herbal | | Water |
| ple | | ular | Amount | Amount | | Amount | Amount |
| Number | Type | Weight | (wt. %) | (wt. %) | Type | (wt. %) | (wt. %) |
| 1 | XPVP | | 32 | 58 | | 0 | 10 |
| 2 | XPVP | | 32 | 54 | CZ | 5 | 9 |
| 3 | XPVP | | 32 | 32 | CZ | 5 | 31 |
| 4 | XPVP | | 32 | 13 | CZ | 5 | 50 |
| 5 | XPVP | | 32 | 54 | BY | 5 | 9 |
| 6 | XPVP | | 30 | 51 | BY | 10 | 9 |

Examples 1-6 were evaluated for mechanical properties by using a TA-XT2i Texture Analyzer (commercially available from Texture Technologies Corp., Scarsdale, N.Y.). A TA57 R (Diameter=10 mm) stainless steel probe was used in compression mode at room temperature. The Compression Force and Pull Force were measured and recorded in grams. The results are shown in Table 2.

TABLE 2

| Example | Compression Force | Pull Force | Subjective |
|---|---|---|---|
| Number | (g) | (g) | Observations |
| 1 | 12 | 39 | Elastic, sticky gel |
| 2 | 27 | 93 | Elastic, sticky gel |
| 3 | 15 | 77 | Elastic, sticky gel |
| 4 | 11 | 42 | Elastic, sticky gel |
| 5 | Not available | Not available | Elastic, sticky gel |
| 6 | Not available | Not available | Elastic, sticky gel |

Herbal release profiles were evaluated using an Agilent 1100 liquid chromatograph (Agilent technologies, Wilmington, Del.). A reversed-phase separation on Zorbax cyano column (150×4.6 mm ID) by acetonitrile/water mobile phase was used for the analysis. The ACN gradient is from 5 to 65% in 40 minutes at a flow rate of 1 mL/min. The detection is 50 μL. Peaks appearing at a retention time of about 25 minutes were used to calculate CZ herbal release. Peak areas appearing at about 16 minutes were used to calculate BY herbal release.

0.4 g of the material was submerged in water in a jar. The jar was placed on a shaker. The water solution was analyzed at various time intervals. for using an Agilent 1100 liquid chromatograph (Agilent technologies, Wilmington, Del.). A reversed-phase separation on Zorbax cyano column (150×4.6 mm ID) by acetonitrile/water mobile phase was used for the analysis. The ACN gradient is from 5 to 65% in 40 minutes at a flow rate of 1 mL/min. The detection is 50 μL. Peaks appearing at a retention time of about 25 minutes were used to calculate CZ herbal release. Peak areas appearing at about 16 minutes were used to calculate BY herbal release.

FIGS. 3 and 4 show the CZ and BY herbal release profile for an adhesive composition of the present invention. The release curve plateaued after 0.5-1.0 hours which indicated that the herbal ingredients were released relatively quickly from the hydrogel composition. Also, there was no significant interference or interaction between herbal ingredients and the adhesive composition.

### Comparative Example 7

Comparative Example A is a commercial brand plaster, Yun-Nan Bai-Yiao (BY) plaster, from Yun-Nan Bai-Yiao Group Co., LTD, Yun-Nan, China. The plaster was made of rubber-based pressure sensitive adhesive mixed with 10% herbal ingredients. An herbal release profile was calculated for Comparative Example 7 as described for Examples 1-6. FIG. 4 shows a comparison of hydrophilic herbal release from the hydrophilic gel adhesive in Examples 5 and 6 to the hydrophobic adhesive in Comparative Example 7.

Examples 5 and 6 reached the peak of 40-45% total herbal release in about 4 hours while Comparative Example 7 reached only 2% of total herbal release after 8 hours. The results indicated that the hydrophilic gel or adhesive of Examples 5 and 6 was a better reservoir and gave better herbal release for hydrophilic herbals than the hydrophobic adhesive of Comparative Example 7.

### Examples 8-11

Herbal medicine compositions were prepared using the components and amounts shown in Table 3. Examples 8-11 were prepared by premixing YPFS herbal powder with glycerin and deionized water. The polymer materials were charged into a Ross double plannary mixer equipped with HV blades and stirred for 5 min. under vacuum. The herbal solution was introduced into the mixer, and the mixture was stirred for 15 min. under vacuum. The pastes were poured between two release liners and compressed with a press with a gauged thickness of 0.5 mm for 3 minutes.

TABLE 3

| | XPVP | Glyerin | HPG | YPFS | Water |
|---|---|---|---|---|---|
| Example | Amount | Amount | Amount | Amount | Amount |
| Number | (parts) | (parts) | (parts) | (parts) | (parts) |
| 8 | 30 | 59.5 | 0 | 5 | 10.5 |
| 9 | 25 | 59.5 | 5 | 5 | 10.5 |
| 10 | 20 | 59.5 | 10 | 5 | 10.5 |
| 11 | 15 | 59.5 | 15 | 5 | 10.5 |

The herbal compositions were evaluated for adhesive properties. Peel adhesion and T-peel tests were performed using a Thwing-Albert EJA-Material Tester (commercially available from Twang-Albert Co., Philadelphia, Pa.). For the peel adhesion test, the hydrogel was cut into 2.54 cm by 5.08 cm strips and laminated between aluminum foil by hand using a 2 kg roller. The strips were peeled off after 24-hour dwell time.

For the T-peel test, the hydrogel strips were first laminated between two paper scrims. Masking tapes were then laminated on the back of the paper scrims. After 18 hours, the samples were tested using a Crosshead speed of 12 in/min or 30.48 cm/min. The results are reported in Table 4 in grams/2.54 cm.

TABLE 4

| | T-peel strength | Peel adhesion |
|---|---|---|
| Example Number | (g/2.54 cm) | (g/2.54 cm) |
| 8 | 1166 | 946¹ |
| 9 | 1420 | 980 |
| 10 | 1690 | 880 |
| 11 | 2339 | 76 |

¹Adhesive failure was cohesive failure.

The results in Table 4 show that increasing the level of the second modifying polymer, HPG, reduces peel adhesion but significantly increases the cohesive strength of the adhesive composition.

### Example 12

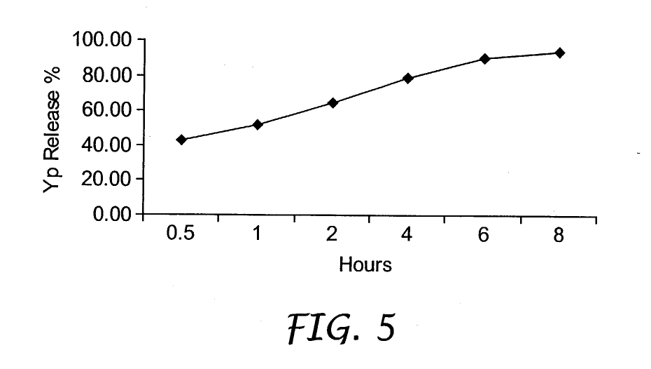

Material from example 10 was analyzed for herbal release activity. The herbal release profile was evaluated using an Agilent 1100 liquid chromatograph (Agilent technologies, Wilmington, Del.). A reversed-phase separation on Zorbax cyano column (150×4.6 mm ID) by acetonitrile/water mobile phase was used for the analysis. The ACN gradient is from 5 to 65% in 40 minutes at a flow rate of 1 mL/min. The detection is 50 μL. Peaks at 5.7, 6.3, 67, 6.9, and 13 min were used. The results indicated the herbal components were readily released as shown in FIG. 5. Also, there was no significant interference or interaction between herbal ingredients and the adhesive composition with the modifying polymer.

1. A hydrophilic adhesive composition comprising:
a swellable adhesive polymer;
a swelling agent; and
herbal medicines;
the swellable adhesive polymer forms a pressure sensitive adhesive in the presence of the swelling agent; and
the herbal medicine is more soluble in the swelling agent than in water alone.

2. The adhesive composition of claim 1, further comprising a modifying polymer, wherein the modifying polymer and the swelling agent to at least maintain the cohesion of the composition.

3. The composition of claim 1 further comprising a therapeutic agent.

4. The composition of claim 1, wherein the swellable adhesive polymer comprises a polymer or copolymer selected from the group consisting of poly (N-vinyl lactam), polyethylene oxide, poly (N-vinyl pyrrolidone), polyacrylamide, maleic anhydride-vinyl ether copolymers, polyacrylic acid, ethylene-maleic anhydride copolymers, polyvinyl ether, polyethyleneimine, polyvinyl alkyl pyridinium halides, polymethacrylic acid and blends of the above.

5. The composition of claim 1 wherein the swellable adhesive polymer comprises lactam poly (N-vinyl lactam).

6. The composition of claim 1 wherein the poly (N-vinyl lactam) is selected from the group consisting of poly N-vinyl-2-pyrrolidone, poly N-vinyl-2-valerolactam, poly N-vinyl-2-caprolactam, and combinations of the foregoing.

7. The composition of claim 1 wherein the swelling agent is selected from the group consisting of monohydric alcohols; polyhydric alcohols; glycerol; polyglycerols; sorbitol; polyhydric alcohol ethoxylates; methoxides of polyethylene glycol; methoxides of polyhydric alcohol ethoxylates; and combinations of the foregoing.

8. The composition of claim 7 wherein the monohydric alcohols consist of ethanol, isopropanol, n-propanol, and combinations of the foregoing.

9. The composition of claim 7 wherein the polyhydric alcohols consist of propylene glycol, dipropylene glycol, polyethylene glycol, glycerin, and combinations of the foregoing.

10. The composition of claim 9 wherein the polyethylene glycol is of a molecular weight between 200 and 600.

11. The composition of claim 7 wherein the polyglycerols consist of diglycerin, triglycerol, polyglycerin-3, hexaglycerol, decaglycerol and combinations of the foregoing.

12. The composition of claim 9 wherein the polyhydric alcohol ethoxylates consist of sorbeth-6, sorbeth-30, glycereth-1 to glycereth-31.

13. The composition of claim 7 wherein the methoxides of polyethylene glycol consist of methoxy polyethylene glycol-2 to methoxy polyethylene glycol-100.

14. The composition of claim 7 wherein the methoxides of polyhydric alcohol ethoxylates is glycereth-7 methoxide.

15. The adhesive composition of claim 1, wherein the swelling agent is essentially non-volatile.

16. The adhesive composition of claim 15, wherein the swelling agent is present in the composition greater than 30%.

17. The adhesive composition of claim 2 wherein the modifying polymer comprises a polysaccharide, polysaccharide derivatives, acrylate, acrylate derivates, collagen, collagen derivatives, cellulose, cellulose derivatives, poly vinyl alcohol, and combinations thereof.

18. The composition of claim 17 wherein the modifying polymer is selected from the group consisting of hydroxypropyl guar; guar gum; hydroxyethyl cellulose; hydroxypropyl cellulose; hydroxypropyl methylcellulose; polymeric quaternary ammonium salt of hydroxyethyl cellulose reacted with trialkyl ammonium substituted epoxide; copolymers of hydroxyethyl cellulose and diallyldimethyl ammonium chloride; and derivatives and combinations of the foregoing.

19. The composition of claim 2 wherein: the swellable adhesive polymer is present in the composition in an amount between 10% and 50% by weight; the swelling agent is present in an amount of at least 55% by weight; and the modifying polymer is present in an amount between 0.1% and 40% by weight.

20. The composition of claim 1, wherein the herbal medicine is present in an amount up to 10% by weight.

21. The composition of claim 2 wherein swellable adhesive polymer is poly N-vinyl-2-pyrrolidone; the swelling agent is glycerin; and the modifying polymer is selected from the group consisting of hydroxypropyl guar; guar gum; hydroxyethyl cellulose; hydroxypropyl cellulose; hydroxypropyl methylcellulose; polymeric quaternary ammonium salt of hydroxyethyl cellulose reacted with trialkyl ammonium substituted epoxide; copolymers of hydroxyethyl cellulose and diallyldimethyl ammonium chloride; and derivatives and combinations of the foregoing.

22. A medical article, comprising a backing layer and the adhesive composition of claim 1.

23. The medical article of claim 22, further comprising a modifying polymer, wherein the modifying polymer and the swelling agent at least maintain the cohesion of the composition.

24. A method for the manufacture of the adhesive composition of claim 2, the method comprising
(a) mixing an uncrosslinked or a partially crosslinked precursor of the swellable adhesive polymer with the swelling agent and the modifying polymer; and
(b) irradiating with gamma radiation the precursor of the swellable adhesive polymer to cross-link the precursor and provide the composition of claim 2.

25. A method for the manufacture of the adhesive composition of claim 2, the method comprising
(a) irradiating with gamma radiation the precursor of the swellable adhesive polymer to cross-link the precursor;
(b) mixing the crosslinked swellable adhesive polymer with the swelling agent and the modifying polymer to provide the composition of claim 2.

26. The adhesive composition of claim 1, wherein the swelling agent is greater than 50% of the total weight of the composition.

27. The adhesive composition of claim 1, wherein the swelling agent comprises glycerin.

28. The medical article according to claim 22, wherein the backing layer comprises a film, substrate, or elastic, porous or breathable woven or nonwoven material.

29. The medical article according to claim 22, wherein the article comprises a medical tape, a wound dressing, a bandage or a medical skin covering.

30. A transdermal delivery device comprising: an adhesive layer for contacting skin and a backing layer, the adhesive layer adhered to the backing layer and comprising the adhesive composition of claim 1.

31. The delivery device according to claim 30, wherein the adhesive layer further comprises a topical, transdermal, or iontophoretic therapeutic agent.

32. The delivery device according to claim 30, wherein the adhesive layer further comprises an excipient, a solvent, or a penetration enhancing agent.

33. The composition according to claim 4, wherein the poly(N-vinyl lactam) is poly(N-vinyl pyrrolidone) and the amount of the swelling agent ranges from about 60 to about 80 weight percent of the composition.

34. The composition according to claim 33, wherein the poly(N-vinyl lactam) is poly(N-vinyl-2-pyrrolidone) homopolymer.

35. The composition according to claim 33, wherein the poly(N-vinyl lactam) is a copolymer of N-vinyl-2-pyrrolidone monomer and a non-N-vinyl lactam comonomer selected from the group consisting of N,N- dimethylacrylamide, acrylic acid, methacrylic acid, hydroxyethylmethacrylate, acrylamide, vinyl acetate, and 2-acrylamido-2-methyl-1-propane sulfonic acid or its salt; the copolymer comprising N-vinyl-2-pyrrolidone monomeric units in an amount no less than about 50 weight percent of the poly(N-vinyl lactam).

36. The composition according to claim 35, wherein the copolymer comprises N-vinyl-2-pyrrolidone monomeric units in an amount from about 70 to about 100 weight percent of the poly(N-vinyl lactam).

37. The composition according to claim 34, wherein the crosslinked poly(N-vinyl lactam) has a Swelling Capacity of at least 15 milliliters of water per gram of the radiation-crosslinked poly(N-vinyl lactam).

38. The composition according to claim 4, wherein the cross-linked poly(N-vinyl lactam) is radiation-crosslinked while in solid form.

39. The composition according to claim 2, wherein the herbal medicine, the modifying polymer and the swelling agent are present in the composition in unirradiated form.
